(19) United States
(12) Patent Application Publication
(10) Pub. No.: US 2002/0048808 A1
(43) Pub. Date: Apr. 25, 2002

(54) BIODEGRADATION OF ETHERS USING A BACTERIAL CULTURE

(76) Inventor: JOSEPH PATRICK SALANITRO, HOUSTON, TX (US)

Correspondence Address: Y GRACE TSANG, SHELL OIL COMPANY, LEGAL - INTELLECTUAL PROPERTY, PO BOX 2463, HOUSTON, TX, 77252-2463, US

(21) Appl. No.: 09/438,595

(22) Filed: Nov. 12, 1999

Related U.S. Application Data
(63) Continuation-in-part of application No. 09/292,037, filed on Apr. 14, 1999, now Pat. No. 6,238,906.
Application No. 09/292,037 is a continuation-in-part of application No. 08/465,996, filed on Jun. 6, 1995, now Pat. No. 5,750,364.

(51) Int. Cl. C12P 39/00; C12S 1/00; B09B 3/00; C02F 9/00

(52) U.S. Cl. 435/262.5; 210/262

(57) ABSTRACT

A bacterial culture capable of degrading ethers, especially branched alkylethers including MTBE, under aerobic conditions has been prepared.

This is a continuation-in-part of application Ser. No. 09/292,037, filed Apr. 14, 1999, which is a continuation-in-part of application Ser. No. 465,996, filed Jun. 6, 1995.

### FIELD OF THE INVENTION

This invention relates to a process for degrading ethers, such as methyl t-butyl ether (MTBE), utilizing a bacterial culture. This invention further relates to a bacterial culture capable of degrading ethers, including methyl t-butyl ether (MTBE), and the process for preparing such culture.

### BACKGROUND OF THE INVENTION

Alkyl-alkyl ethers (R—O—R) such as methyl t-butyl ether (hereinafter “MTBE”) are being used as octane-enhancers in the reformulation of low volatility unleaded gasoline blends and for reducing the emission of volatile organic compounds from engines. In general, alkylethers, especially those alkylethers which have only one ether linkage and without other functional groups, are chemically stable compounds and there is little information on their biodegradability in soil, groundwater and activated sludge environments. The lack of alkylether degradation by indigenous microbes in soils and biosludges may be attributed to the very stable and chemically unreactive ether linkage, the inability of these compounds to be transported into cells and/or the lack of inducible or existing enzyme activities (e.g. oxygenases, hydroxylases) which can attack the ether bond.

It is known that MTBE can persist in groundwater from accidental spills of unleaded gasoline from underground storage tanks. However, no known naturally occurring microbial cultures exist to effectively biotreat groundwater, wastewater, tank bottom wastes or soils containing this ether.

Alkyl ethers such as symmetric dioctyl ether have been shown by Modrzakowski and Finnerty to be only partially oxidized by an Acinetobacter strain in which the ether linkage is not cleaved and only the terminal carbons are utilized for growth. See, Intermediary Metabolism of Acinetobacter Grown on Dialkyethers. Can. J. Microbiol., 35:1031-1036 (1989).

Studies on the biodegradability testing of MTBE in sludges and soils by Fujiwara et al. showed that 100 ppm MTBE or diisopropylether (DIPE) does not degrade in activated sludge (300 ppm solids) in an oxygen uptake assay. Moreover, MTBE did not significantly affect the respiration rate of other hydrocarbons when blended (12% w/v) with the fuel. See, Fujiwara, T., T. Kinoshita, H. Sato and I. Kojima, Biodegradation And Bioconcentration of Alkyl Ethers, Yukagaku 33:111-114 (1984).

Moller and Arvin proposed that MTBE (10 ppm) or TAME (t-amyl methyl ether, 3 ppm) were not degraded in 60 days by microbes in an aquifer soil, topsoil or activated sludges. In these experiments, MTBE at 200 ppm levels showed a weak inhibitory effect on the biodegradation of aromatic hydrocarbons (3.5 ppm BTEX). See, Moller, H. and E. Arvin, Solubility and Degradability of The Gasoline Additive MTBE, Methyl-tert-butyl-ether and Gasoline Compounds in Water, Contaminated Soil '90, 445-448 (1990), Kluwer Academic Publishers.

Recent studies by Suflita and Mormile on the anaerobic degradation of gasoline oxygenates in a landfill aquifer material showed that of several alkyl ethers tested (MTBE, TAME, ETBE, DIPE, ethyl ether, propyl ether) only n-butyl methyl ether was metabolized under anaerobic methanogenic conditions. MTBE is only cleaved under anaerobic condition to t-butyl alcohol which is not degraded further. See Suflita, J. M. and M. R. Mormile, Anaerobic Biodegradation of Known and Potential Gasoline Oxygenates in the Terrestrial Subsurface, Environ. Sci. Technology 27:976-978 (1993).

Parales et al isolated an actinomycete from biosludge which was shown to grow on 1,4-dioxane could also utilize some of the linear alkyl ethers such as diethyl ether and methyl butyl ether, but not the branched alkyl ethers such as diisopropylether, ethyl t-butyl ether or ethylene glycol ethers. See, Parales, R. E., J. E. Admus, N. White, H.D. Degradation of 1,4-dioxane by an Actinomycete in Pure Culture, Applied Environ Microbiol, 60, 4527-4530, May, 1994.

Japanese patent application number 04,110,098, filed by Kyowa Hakko Kagyo KK, proposes the decomposition of ethylene glycol alkylethers with bacteria. The ethers decomposed have more than one ether linkages and/or have hydroxyl functional groups, which are known to be more readily degradable than those with only one ether linkage and without other functional groups.

Japanese patent application number 62,208,289, filed by Hodogaya Chem. Ind. KK, proposes the degradation of polyoxytetramethylene glycol with bacterial strains. The ethers degraded have multiple ether linkages and thus are more readily degradable than those with only one ether linkage and with no other functional groups.

Thus, there remains a need for a bacterial culture capable of degrading under aerobic condition an ether, especially an alkyl ether, more especially a branched alkyl ether such as MTBE. The culture would be useful for treating wastes and groundwater containing ethers, especially branched alkyl ethers such as MTBE.

### SUMMARY OF THE INVENTION

This invention relates to (a) a bacterial culture capable of degrading alkylethers, especially branched alkylethers including MTBE, under aerobic conditions; (b) a process for preparing a bacterial culture which is capable of degrading alkylethers, especially branched alkylethers such as MTBE, to CO₂ using activated sludges; (c) a process for the aerobic degradation of ethers, especially branched alkylethers such as MTBE, using a bacterial culture prepared from activated sludges; (d) a process for remediating wastes and groundwater containing ethers, especially branched alkylethers such as MTBE, to reduce the alkylether(s) content thereof by growing in the presence of said wastes and groundwater a population of a bacterial culture prepared from activated sludges, particularly a pure bacterial culture; specifically a population of a culture derived from a mixed bacterial culture, more specifically a pure culture derived from a mixed bacterial culture. This invention further relates to a pure bacterial culture which degrades alkylethers, especially branched alkylethers including MTBE, under aerobic conditions to carbon dioxide and water; and a process for preparing such a pure bacterial culture from said mixed bacterial culture. This invention also relates to the use of said pure culture for degrading or remediating t-butyl alcohol containing aqueous solutions or groundwater.

### DETAILED DESCRIPTION OF THE INVENTION

A culture of BC-1 has been deposited with the American Type Culture Collection (ATCC), Patent Depository, 12301 Parklawn Drive, Rockville, Md. 20852 with ATCC number 202057. A culture of BC-1, ATCC number 20205, can be obtained from the permanent collection of the ATCC, Patent Depository. A pure culture derived from the present mixed culture will be deposited with American Type Culture Collection (ATCC), Patent Depository, 12301 Parklawn Drive, Rockville, Md. 20852 promptly after the allowance of the instant patent application with ATCC number ______. Upon issuance of the subject application as a patent, pure culture of the present invention can be obtained from the permanent collection of the American Type Culture Collection (ATCC), Patent Depository, 12301 Parklawn Drive, Rockville, Md. 20852.

The present invention relates to a bacterial culture capable of degrading a branched alkyl ether. Specifically, the present invention involves a pure bacterial culture capable of degrading aerobically a branched alkyl ether, particularly a tertiary carbon atom-containing alkyl ether, more particularly MTBE, to CO₂ which is derived from a mixed bacterial culture. The invention also relates to a mixed bacterial culture which degrades aerobically a branched alkyl ether, particularly a tertiary carbon atom-containing alkyl ether, more particularly MTBE, to CO₂. The bacterial culture(s) is capable of cleaving the ether linkage of methyl t-butyl ether (MTBE) with the transient formation of t-butyl alcohol (TBA) and degrading completely to CO₂. The novel bacterial culture can also metabolize other linear and branched ethers. Non-limiting and illustrative examples of the linear and branched ethers include diethyl ether (DEE), dimethyl ether (DME), methyl ethyl ether (MEE), methyl n-propyl ether (MPE), ethyl n-propyl ether, methyl isopropyl ether, ethyl isopropyl ether, diisopropyl ether (DIPE), ethyl t-butyl ether (ETBE) or methyl-t-amyl ether. Specifically, the invention relates to a novel mixed bacterial culture, designated BC-1 with ATCC No. 20257 which is capable of degrading MTBE completely to CO₂ with the transient formation of t-butyl alcohol (TBA).

As a more specific embodiment of the present invention, the novel mixed bacterial culture includes any composition derived from the mixed bacterial culture enriched from incubating activated sludge and a branched alkyl ether. Illustrative examples of the compositions derived from the mixed bacterial culture include, but not limited to, members of, fragments of bacterial culture, membrane fragments of bacterial culture, enzymes extracted and/or isolated from the bacterial culture, lyophilized and/or dried culture, lyophilized and/or dried fragments of culture, lyophilized and/or dried enzymes derived from said culture, bacterial culture and/or fragments thereof and/or enzymes derived therefrom bound to a carrier and/or binder and/or fixed bed, etc. Any method known to one skilled in art for making composition derived from the mixed culture including but not limited to extraction or fragmentation to obtain active ingredients/fragments thereof is within the scope of the present invention. As one non-limiting example of the present invention, the mixed culture can be first fragmented by sonification or lysing with lysozyme and/or a compound such as a chelating compound, followed by salting out the enzyme fractions using ammonium sulfate or NaCl.

As one specific aspect of the aforementioned embodiment of the present invention, the composition derived from said mixed bacterial culture is a pure bacterial culture isolated from said mixed bacterial culture. As another specific embodiment of the present invention, the pure culture isolated belongs to the family Actinomycetes. As still another specific embodiment of the present invention, the pure culture isolated is a Rhodococcus species. As yet another specific aspect of the aforementioned embodiment of the present invention, the composition derived from said mixed bacterial culture is a composition derived from a pure culture isolated from the present mixed culture. Illustrative examples of the compositions derived from the pure bacterial culture include, but not limited to, member(s) of, fragment(s) of the bacterial culture, membrane fragments of bacterial culture, enzymes extracted and/or isolated from the bacterial culture, lyophilized and/or dried culture, lyophilized and/or dried fragments of culture, lyophilized and/or dried enzymes derived from said culture, bacterial culture and/or fragments thereof and/or enzymes derived therefrom bound to a carrier and/or binder and/or fixed bed, etc. Any method known to one skilled in art for making composition derived from a culture including but not limited to extraction or fragmentation to obtain active ingredients/fragments thereof is within the scope of the present invention.

As a particular embodiment of the present invention, the present culture, derived from said mixed bacterial culture is a pure culture strain having the identifiable characteristics of a pure culture with ATCC No. ______. As used herein, the term “identifiable characteristic of the pure culture with ATCC No. ______” means the ability to degrade MTBE; specifically the ability to degrade MTBE in 70 hours; more specifically the ability to degrade at least 10% of the MTBE present in a MTBE-containing mixture to carbon dioxide in 70 hours; even more specifically the ability to degrade both MTBE and TBA, preferably to carbon dioxide in 70 hours; more specifically the ability to degrade at least 10% of the MTBE and/or TBA added to the culture at a concentration of 0.01 to 500 ppm, to carbon dioxide within 70 hours; particularly the ability to degrade to carbon dioxide MTBE and one or more of the following ether compounds: diisopropyl ether, ethyl-t-butyl ether, di-t-butyl ether, diisobutyl ether, isopropyl isobutyl ether, isopropyl t-butyl ether, t-amylmethyl ether, t-amylethyl ether, t-amyl ethyl ether, t-amyl propyl ether, t-amylisopropyl ether, t-amyl-n-butyl ether, t-amyl isobutyl ether, and t-amyl methyl ether within 70 hours; more particularly the ability to degrade to carbon dioxide MTBE and one or more of the following tertiary carbon-containing ether compounds: ethyl-t-butyl ether, t-amyl-n-butyl ether, t-amylisobutyl ether, isopropyl t-butyl ether, t-amyl ethyl ether, t-amylpropyl ether, t-amylisopropyl ether, and methyl t-amyl ether within 70 hours.

As one specific embodiment of the present invention, the specific activity of the present pure culture is from about 0.1 to about 100, preferably from about 1 to about 50, more preferably from about 5 to about 30 mg MTBE/g cells/hr at 9° C.

As a specific embodiment of the present invention, the present pure culture is a pure culture having ATCC No. ______. As another specific embodiment of the present invention, the present pure culture is derived or isolated from the present mixed culture, specifically a mixed culture have the identifying characteristics of the BC-1 culture with ATCC Number 202057. As used herein, the term “identifiable characteristic of the mixed culture BC-1 with ATCC No. 202057” means the ability to degrade MTBE; specifically the ability to degrade MTBE in 70 hours; more specifically the ability to degrade at least 10% of the MTBE present in a MTBE-containing mixture to carbon dioxide in 70 hours; even more specifically the ability to degrade both MTBE and TBA, preferably to carbon dioxide in 70 hours; more specifically the ability to degrade at least 10% of the MTBE and/or TBA added to the culture at a concentration of 0.01 to 500 ppm, to carbon dioxide within 70 hours; particularly the ability to degrade to carbon dioxide MTBE and one or more of the following ether compounds: diisopropyl ether, ethyl-t-butyl ether, di-t-butyl ether, diisobutyl ether, isopropyl isobutyl ether, isopropyl t-butyl ether, t-amylmethyl ether, t-amylethyl ether, t-amyl ethyl ether, t-amyl propyl ether, t-amylisopropyl ether, t-amyl-n-butyl ether, t-amyl isobutyl ether, and t-amyl methyl ether within 70 hours; more particularly the ability to degrade to carbon dioxide MTBE and one or more of the following tertiary carbon-containing ether compounds: ethyl-t-butyl ether, t-amyl-n-butyl ether, t-amylisobutyl ether, isopropyl t-butyl ether, t-amyl ethyl ether, t-amylpropyl ether, t-amylisopropyl ether, and methyl t-amyl ether within 70 hours.

The present invention also relates to a process for preparing a pure bacterial culture capable of degrading branched ether from a mixed bacterial culture capable of degrading the same. As a particular aspect of the present invention, it is provided with a process for isolating a pure culture from a mixed bacterial culture. Any method known to one skilled in the art which is able to isolate the MTBE-degrading pure culture from a mixed culture capable of degrading branched ether such as MTBE is within the scope of the present process. Non-limiting example of the process suitable for isolating the pure culture(s) of the present invention includes enhancing isolation of the pure microbe(s) capable of degrading MTBE by first making dilution enrichments of the present mixed culture(s) . As an illustrative example, the dilution enrichments are made by adding sterile mineral dilution medium(such as Bushnell-Haas minerals (BH)-Difco-sterile) containing about 0.01-1000 mg/L, specifically about 0.1-100 mg/L, more specifically from about 1-10 mg/L MTBE to the mixed culture at about from 5:1 to 0.2:1, specifically from about 3:1 to about 0.3:1 ratio, more specifically at about 1.5:1 to 1:1.5 ratio. At certain time intervals such as weekly, biweekly or monthly, a portion of the culture volume was aseptically removed and replaced with fresh sterile dilution medium added to the remaining culture. The dilution enrichment method was continued for an extended period of time of about 2-60 weeks, specifically of about 4-25 weeks, and more specifically of about 7-14 weeks at about 10-40° C., specifically about 20-35° C. and more specifically at 23° C. to 32° C. until a dilute suspension of bacteria degrading MTBE consistently degraded MTBE before each transfer interval. This dilution enriched culture can subsequently be streaked onto sterile Petri plates containing minerals and solidifying agent such as 1.5% Difco Agar. Plates were incubated at about 10-40° C., specifically about 20-35° C. and more specifically at 23° C. to 32° C. and observed for the appearance of colonies after about 1-70 days, specifically after 2-50 day, more specifically after 3-5 days. All of the colonies were individually picked with sterile needles and inoculated containers/vials containing sterile mineral medium (such as BH medium) containing about 0.01-1000 mg/L, specifically about 0.1-100 mg/L, more specifically from about 1-10 mg/L MTBE. The cultures were incubated at about 10-40° C., specifically about 20-35° C. and more specifically at 23° C. to 32° C. and the loss of MTBE from the headspace of containers/vials was determined. Isolate(s) which degrade MTBE effectively preferably without the appearance of intermediates such as t-butyl alcohol are identified as pure culture capable of degrading MTBE and preferably also TBA effectively.

One of the mixed cultures suitable for preparing the present pure culture is available as product BC-1 from Shell Oil Company and its affiliate Equilon Enterprises LLC in Houston, Tex.

As one embodiment of the present invention, The isolated pure bacteria culture can be grown to obtain a larger population of a larger quantity of the culture by growing in a sugar (such as glucose) containing BH mineral solution. As another embodiment of the present invention, the isolated pure culture is grown in an MTBE-containing mineral media. Particularly, the culture is grown in a sugar containing mineral solution and the MTBE degrading activity is induced by adding MTBE to the culture. As a specific aspect of this embodiment, the MTBE degrading activity, i.e. the capability of degrading MTBE (the concentration of the MTBE degraded within about five hours, specifically about 10 hours, more specifically about 50 hours) is increased by at least 25%, preferably by at least 50%, more preferably by at least 100% after incubating with 1-1000 mg/L, specifically about 5-500 mg/L, more specifically about 20-200 mg/L for less than 10 hour, specifically for about less than 20 hours, and more specifically for less than 50 hours.

As still another aspect of the present invention, the specific activity of the present pure culture is from about 0.1 to about 100, preferably from about 1 to about 50, more preferably from about 5 to about 30 mg MTBE/L at 9° C.

The present invention further relates to a mixture of the present pure culture and the present mixed culture.

The present invention also relates to a process for preparing the above-mentioned novel mixed bacterial culture by adding a branched alkyl ether such as MTBE to an activated sludge, specifically activated sludge obtained from chemical plant, petrochemical plant or a refinery, more specifically from a biotreater located in a wastewater treatment plant in a refinery or a petrochemical plant. As a specific embodiment of the present invention, the activated sludge is retrieved from the biotreater located in a wastewater treatment plant of a chemical plant. As a still more specific embodiment of the present invention, the activated sludge is retrieved from the biotreater of the South Effluent Treater for treating wastewater from the Chemical Plant of Shell Deer Park Manufacturing Complex located at 5900 Highway 225, Deer Park Tex. 77536.

The mixed culture is prepared by adding a branched alkyl ether to the biosludge (activated sludge) and incubating for a period time. As one specific embodiment of the present invention, the biosludge is first added to a mineral nutrient solution. One specific, but non-limiting, example of the mineral solution is Sturm solution comprising KH₂PO₄, K₂HPO₄, Na₂HPO₄.2H₂O, MgSO₄.7H₂O, NH₄Cl, (NH₄)₂ SO₄, and FeCl₃.6H₂O. Incubation using other nutrient solution known to those skilled in the art is within the scope of the present invention. The concentration of the biosludge in the incubated medium (culture) can be any suitable amount which would produce sufficient concentration of ether degrading bacteria. In a specific embodiment of the present invention, from about 50 mg to about 5000 mg, more specifically from about 50 mg to about 1500 mg, still more specifically from about 300 to about 800 mg, of the biosludge solids are added to every liter of the incubation medium.

The above mixed culture is enriched by adding a suitable amount of branched alkyl ether. In a specific embodiment of the present invention, about 5-5000 mg, more specifically about 10-500 mg, still more specifically about 30-50 mg, of the branched alkyl ether is added to every liter of the culture (incubation medium or mixture).

The mixture or culture is incubated for a period of time. The typical temperature at which the culture is incubated ranges from about 5° C. to about 80° C., specifically from 10° C. to about 60° C., more specifically from about 15° C. to about 35° C., still more specifically from about 22° C. to about 25° C. Periodically, a sample of the culture (or supernatant) is withdrawn for branched alkyl ether analysis. A culture is active in degrading branched alkyl ether if there is detectable reduction of the concentration of the branched alkyl ether in the culture being enriched, after taken into account of the amount of branched alkyl ether evaporated. As an illustrative but non-limiting example, a culture which is considered very active in degrading branched alkyl ether will degrade a solution containing about 0.001-5000 ppm, more specifically about 0.01-500 ppm, still more specifically about 0.05-100 ppm, of branched alkyl ether by from about 10% to about 100%, specifically from about 30% to about 100%, more specifically from about 50% to about 100%, still more specifically from about 80% to about 100% in from about 2 hours to about 70 hours, specifically from about 2 hours to about 12 hours, more specifically from about 3 hours to about 5 hours. As an illustrative non-limiting example, a culture of the present invention is capable of degrading a solution containing 120 mg/L of MTBE to close to 0 mg/L of MTBE in about 4 hours or less.

In one specific embodiment of the present invention, the mixture of the activated sludge and the mineral solution is first flushed with oxygen before the addition of the branched alkyl ether.

In still another specific embodiment of the present invention, periodically, a portion in an amount of about 5-80%, specifically about 10-70%, more specifically about 40-60%, of the supernatant of the culture is withdrawn and fresh mineral or nutrient solution is added to at least partially replace the amount of supernatant withdrawn. The withdrawal can be conducted at an interval of about 1-30 days, specifically 2-10 day, more specifically about 5-8 days.

As another specific embodiment of the present invention, multiple additions of branched alkyl ether are subsequently made to the culture (incubating medium) after the first addition of the branched alkyl ether. The subsequent additions were made at least two days after the first addition of the branched alkyl ether. As a specific aspect of this embodiment, sufficient amount of branched alkyl ether is added either immediately after each withdrawal of the supernatant or simultaneously with the addition of the replacement portion of mineral or nutrient solution, thereby compensating the loss of the branched alkyl ether resulted from the withdrawal. As another specific aspect of this embodiment, sufficient alkyl ether is added each time designed to maintain the alkyl ether concentration at about 50-150%, specifically about 80-120%, of the original concentration.

As a preferred embodiment of the present invention, multiple additions (re-inoculation) of the activated sludge is made to the culture periodically, such as at an interval of about 2-60 days, specifically about 3-30 days, more specifically about 5-10 days. In a specific aspect of this embodiment, from about 50 mg to about 5000 mg, more specifically from about 50 mg to about 1500 mg, still more specifically from about 300 to about 800 mg, of biosludge solids are added to every liter of the incubation medium at each re-inoculation.

Illustrative examples of the branched alkyl ether suitable for the enrichment of the culture to produce the culture of the present invention include, but not limited to, MTBE, diisopropyl ether, ethyl t-butyl ether, di-t-butyl ether, diisobutyl ether, isopropyl isobutyl ether, isopropyl t-butyl ether, isopropyl isobutyl ether, t-amyl methyl ether, t-amyl ethyl ether, t-amyl propyl ether, t-amyl isopropyl ether, t-amyl n-butyl ether, t-amyl isobutyl ether, t-amyl methyl ether, ethyl ether etc.

As a preferred embodiment of the present invention, methyl t-butyl ether (MTBE) is used in the enrichment of the bacterial culture to produce a MTBE degradable culture.

The enrichment process typically lasts from about 1 months to about one year, more typically from about 1.5 months to 5 months, more typically from about 2 months to about 4 months.

As a particular aspect of the present invention, it is provided with a process for making a derivative of the present mixed culture by isolating a pure culture from the present mixed bacterial culture. Any method known to one skilled in the art which is able to isolate the MTBE-degrading pure culture from the present mixed culture is within the scope of the present process. Non-limiting example of the process suitable for isolating the pure culture(s) of the present invention includes enhancing isolation of the pure microbe(s) degrading MTBE by first making dilution enrichments of the present mixed culture (s)

As a more preferred embodiment of the present invention, the culture produced is capable of degrading alkyl ethers, specifically branched alkyl ethers, more specifically MTBE, to carbon dioxide. The culture prepared can also be used to degrade t-butyl alcohol, isopropyl alcohol and acetone.

The present invention further involves a process for degrading ethers, including alkylethers and aromatic ethers utilizing the above-mentioned novel mixed culture and/or pure culture by contacting or growing the aforementioned culture or composition derived from the culture with or in a solution containing the ether to be degraded. The alkylethers include branched alkyl ether and linear alkyl ethers. Specifically, the process of the present invention is effective in degrading branched alkyl ether, particularly MTBE. As a specific embodiment of the present invention, the ether to be degraded can be an ingredient in an aqueous solution such as groundwater and wastewater, a solid mixture such as soil, etc. The degradation is preferably conducted under an oxygen-containing atmosphere, such as aerobic conditions. The degradation can be conducted at a temperature from about 5° C. to about 80° C., specifically from about 10° C. to about 60° C., more specifically from about 15° C. to about 35° C., still more specifically at ambient temperature.

As a specific embodiment of the present process, the bacterial culture is used to remediate groundwater and wastewater containing ether, specifically alkyl ether, more specifically MTBE.

It is known that when MTBE-containing fuels are accidentally released to the subsurface, this alkyl ether is the most water soluble and persistent compound in ground water. Other branched alkyl ethers which behave similarly and have also been considered by the oil industry as octane enhancers for motor fuels are diisopropyl ether (DIPE), ethyl tertiary butyl ether (ETBE) and methyl tertiary amyl ether (MTAE). The present invention thus provides an effective biological process for remediating these ethers accidentally released to the subsurface such as groundwater, wastewater and soil. In a specific embodiment of the present invention, the ethers can be completely mineralized to carbon dioxide by a suitable culture prepared by the aforementioned enrichment process. Hence, the remediation process can be substantially free of environmentally undesirable end products.

The present culture is capable of degrading/remediating ether(s), specifically branched alkyl ether(s), more specifically MTBE, in an aqueous mixture containing from about 0.001 ppm to about 5000 ppm, specifically from about 0.01 ppm to about 500 ppm, more specifically from about 0.05 ppm to about 100 ppm of the ether(s); to reduce the content thereof by from about 10% to about 100%, specifically from about 30% to about 100%, more specifically from about 50% to about 100%, still more specifically from about 80% to about 100% in from about 2 hours to about 70 hours, specifically from about 2 hours to about 12 hours, more specifically from about 3 hours to about 5 hours, by growing in the aqueous mixture the culture of the present invention. As a specific embodiment of the present invention, the present pure culture is used in a concentration of from about 50 ppm to about 10,000 ppm, specifically from about 100 ppm to about 5000 ppm, and more specifically from about 1000 ppm to about 4000 ppm when used for remediating or degrading MTBE. As a specific embodiment, the present pure culture, when initially present at a concentration from about 50 ppm to about 10,000 ppm, specifically from about 50 ppm to about 5000 ppm, more specifically from about 300 ppm to about 4000 ppm in an MTBE-containing mixture comprising from about 500 ppm to about 4000 ppm, specifically from about 1000 ppm to about 4000 ppm, more specifically from about 0.001 ppm to about 5000 ppm of MTBE, is capable of degrading the MTBE to carbon dioxide and water by about 10 to 100 percent, preferably by about 50 to 100 percent, more preferably by about 80 to 100 percent, still more preferably by about 90 to 100 percent, still more preferably by about 95 to about 100 percent in less than about 70 hours, preferably in less than about 50 hours, more preferably in less than about 30 hours. As a non-limiting illustrative example, the pure culture is capable of degrading MTBE present initially at about 4 to about 6 mg per liter down to about 0.5 to about 0.001 mg/L, specifically to about 0.1 to about 0.01 mg/L, more specifically to about 0.007 to about 0.004 mg/L in less than about 50 hours, more specifically in less than about 30 hours at a temperature of about 5 to about 35° C.

As another specific embodiment of the present invention, the isolated bacterial enrichment culture, including the composition derived thereof such as pure culture, can cleave the ether linkage of MTBE with the transient formation of t-butylalcohol (TBA). The t-butylalcohol can be degraded by the culture to carbon dioxide. It can also metabolize other linear and branched ethers including diethyl ether (DEE), dimethyl ether (DME), methyl ethyl ether (MEE), methyl n-propyl ether (MPE), ethyl n-propyl ether, methyl isopropyl ether, ethyl isopropyl ether, diisopropyl ether (DIPE), ethyl t-butyl ether (ETBE) or methyl-t-amyl ether (MTAE), etc.

The present invention further relates to a process for aerobically degrading a branched alkyl ether, specifically a tertiary carbon-containing alkyl ether, in a branched alkyl ether-containing mixture, preferably to carbon dioxide. As a specific embodiment, the present process relates to aerobically degrading MTBE in a MTBE-containing mixture, which process comprises growing in the presence of MTBE-containing mixture the present culture, or its derivatives including the present pure culture as well as both the present pure culture and the present mixed culture to reduce the concentration of the MTBE in the mixture to a lower concentration. As one specific embodiment, the aqueous branched alkyl ether-containing mixture comprises from about 0.001 ppm to about 5000 ppm, specifically from about 0.01 ppm to about 500 ppm, more specifically from about 0.05 ppm to about 100 ppm of the ether(s); and the present process reduces the content of branched alkyl ether, specifically MTBE, by from about 10% to about 100%, specifically from about 30% to about 100%, more specifically from about 50% to about 100%, still more specifically from about 80% to about 100% in from about 2 hours to about 70 hours, specifically from about 2 hours to about 12 hours, more specifically from about 3 hours to about 5 hours. As a specific aspect of the present invention, the process degrades the branched alkyl ether, specifically MTBE, to carbon dioxide and water. The present invention further relates to a process for treating groundwater or wastewater containing a branched alkyl ether such as diisopropyl ether, ethyl-t-butyl ether, di-t-butyl ether, diisobutyl ether, isopropyl isobutyl ether, isopropyl t-butyl ether, t-amylmethyl ether, t-amylethyl ether, t-amyl ethyl ether, t-amyl propyl ether, t-amylisopropyl ether, t-amyl-n-butyl ether, t-amyl isobutyl ether, and t-amyl methyl ether.

The present invention further relates to a process for degrading or remediating branched alkyl alcohol, specifically a tertiary-carbon atom-containing alcohol, more specifically t-butyl alcohol in a branched alkyl alcohol-containing mixture such as groundwater and waste water, using the present pure culture or both the present pure culture and the present mixed culture. The alcohol is degraded to carbon dioxide and water. As a specific embodiment, the present process relates to aerobically degrading t-butyl alcohol in a t-butyl alcohol-containing mixture, which process comprises growing in the presence of t-butyl alcohol-containing mixture the present culture, or its derivatives including the present pure culture to reduce the concentration of the t-butyl alcohol in the mixture to a lower concentration. As one specific embodiment, the aqueous branched alkyl ether-containing mixture comprises from about 0.001 ppm to about 5000 ppm, specifically from about 0.01 ppm to about 500 ppm, more specifically from about 0.05 ppm to about 100 ppm of the ether(s); and the present process reduces the content of t-butyl alcohol, by from about 10% to about 100%, specifically from about 30% to about 100%, more specifically from about 50% to about 100%, still more specifically from about 80% to about 100% in from about 2 hours to about 70 hours, specifically from about 2 hours to about 12 hours, more specifically from about 3 hours to about 5 hours.

The invention will be illustrated by the following illustrative embodiments which are provided for illustration purpose only and are not intended to limit the scope of the instant invention.

### ILLUSTRATIVE EMBODIMENTS

The following illustrative embodiments describe typical techniques of the present invention.

### Part A: Derivation of Ether Degradable Culture

### A-I: BC-1 Ether Degradable Mixed Culture Derived from Activated Sludge from Chemical Plant Biotreater

The biosludge (activated sludge) used in this run (A-I) was retrieved from the biotreater of the South Effluent Treater for treating wastewater from the Chemical Plant of Shell Deer Park Manufacturing Complex located at 5900 Highway 225, Deer Park, Tex. 77536. About 100-200 ml of the biosludge (activated sludge) containing about 300 to 800 mg of biosludge solids were added to 1 liter of Sturm solution containing the following minerals (in milligrams per liter, i.e. ppm) to form a culture in a 2-liter stirred glass vessel sealed with Viton O rings: KH₂PO₄, 17; K₂HPO₄, 44; Na₂HPO₄.2H₂O, 67; MgSO₄.7H₂O, 23; NH₄Cl, 3.4; (NH₄)₂ SO₄, 40; FeCl₃.6H₂O, 1. Information on this mineral solution can be found in Sturm, R. N., Biodegradability of Nonionic Surfactants: Screening Test For Predicting Rate And Ultimate Degradation, J. Am. Oil Chem. Soc., 50:159-167 (1973).

The above culture was enriched by first flushing with oxygen for 5 minutes, followed by adding MTBE at an amount of about 30-50 mg MTBE per liter of the culture.

The culture was stirred continuously at room temperature (22-25° C.). At weekly intervals, 1-3 ml of the slurries were withdrawn and allowed to settle (or be filtered). The supernatant and samples (1-3 ml supernatant) withdrawn for MTBE analysis. At each sampling, the culture was enriched by removing 500 ml of supernatant medium and replacing with 500 ml of the sterile minerals solution containing 30-50 ppm MTBE. No significant reduction of MTBE concentration in the supernatants sampled was detected for about two months.

Starting two months after the commencement of the enrichment procedure, re-inoculation involving multiple additions of about 100-200 ml of the above-described activated-sludge retrieved from Shell Deer Park Chemical Plant biotreater was made to the culture about every 7-30 days for about two months. The above-mentioned enrichment procedure of periodic additions of MTBE and withdrawal of the supernatant was also continued.

After two months, this enriched culture became active in consistently degrading MTBE concentrations in the supernatant about 50% to about 100% in about 2-4 hours. This culture was subsequently designated BC-1.

### A-II: Control—1% NaCN

A vessel used as a control was prepared following the enrichment procedure described in A-I above using the same biosludge material, except sufficient NaCN was added so that the culture contains 1% NaCN. NaCN was used as a microbial respiration inhibitor to monitor any ether loss from volatilization.

### Results

The Control (A-II) showed less than 10% loss of ether from volatilization. Mixed culture made from A-I, subsequently designated BC-1 ATCC No. 20257, consistently degraded MTBE.

### Microscopic and Species Characteristics of BC-1 Culture

Microscopic examination of phase-contrast and gram-stained smears of BC-1 showed that it contains gram-positive filamentous species and several gram-negative smaller rod-shaped bacteria. Preliminary identification of colonies isolated on a minerals (Sturm solution) agar medium containing 200 ppm of MTBE indicate that BC-1 contains at least 4-5 organisms including species of coryneforms, Pseudomonas and Achromobacter. All of these isolates utilize acetate, but none have been shown to grow on MTBE as sole source of carbon.

### Part B: Maintenance and Analysis of BC-1 in a Bench Biotreater

The BC-1 culture obtained from A-I above was placed into a four-liter capacity sealed glass vessel for continuous culture maintenance. A similar suspended solids recycle apparatus with aerator (4L) and clarifier (1L) has been described in Salanitro et al, Effects of Ammonia and Phosphate Limitation on the Activated Sludge Treatment of Calcium-Containing Waste, Biotechnol. Bioeng., 25 513-523 (1983), with the exception that pure oxygen was used in place of air to provide aerobic conditions. Dissolved oxygen was monitored with a Leeds and Northrup 7932 meter and probe and maintained at 4-7 mg/liter (ppm) with an oxygen flow rate of 10 ml/min. MTBE (2% solution) was added continuously at a rate of 30-40 ml/day (150-200 mg/liter (ppm)) using a Watson-Marlow (Model 101U) peristaltic pump. The pH was kept at 7.2-7.5 by the infusion of 2 M NaOH solution from a Masterflex® peristaltic pump. The culture was also fed with a minerals solution (4 liters/day) consisting of NaCl (1,000 mb/liter), NH₄CL (380 ppm), KH₂PO₄ (350 ppm), and MgSO₄.7H₂O (30 ppm). The ether-degrading culture developed a stable nitrifying population under high NH₄⁺ (380 mg/liter (ppm) NH₄CL) or low NH₄⁺ (65 mg/liter (ppm) NH₄Cl) conditions. Suspended solids removed from the unit included 35-40 ml/day from the aerator 3.0 (intentionally wasted) and 8 to 48 mg/day from the effluent. This waste rate was equivalent to a 80-90 day cell residence time.

Influent and effluent samples from the continuous biotreater were analyzed for cell dry weight according to methods outlined in Standard Methods For The Examination of Water And Wastewater, 17th Ed. Method 5210-B, American Public Health Association, Washington, D.C. NH₄⁺, NO₃⁻ and PO₄⁻³ ions were estimated by routine Dionex™ ion chromatography.

Data on the growth and metabolism of the BC-1 culture in the solids recycle culture are given in TABLE 1 below.

TABLE 1
Nitrification and Biomass Yields in BC-1
Continuous Culture Degrading MTBE

Nitrifying Condition

| Parameter^{a)} | High NH₄⁺ | Low NH₄⁺ |
|---|---|---|
| Influent NH₄⁺, ppm | 120-125 | 10-20 |
| Effluent NO₃⁻, ppm | 390-450 | 50-70 |
| Reactor TSS, ppm^{b)} | 2500-2580 | 2020-2340 |
| Solids retention, | 80-90 | 80-85 |
| days | | |
| Average % MTBE | 80-90^{c)} | 60-65^{d)} |
| removed | | |
| Cell yield, g TSS/g | 0.21-.24 | 0.23-.28 |
| MTBE utilized | | |

^{a)}Analyses given are the average of four weeks data under each condition.
^{b)}Waste rates were 1.1-1.3 liters every four weeks; effluent TSS under both conditions varied from 2-12 ppm and contributed 25-30% of biomass loss from the unit.
^{c)}Influent and effluent MTBE varied from 160-210 ppm and 3-40 ppm, respectively.
^{d)}Influent and effluent MTBE varied from 120-175 ppm and 50-60 ppm, respectively.

### Part C: Batch Substrate Removal Experiments

The utilization of MTBE and t-butyl alcohol (TBA), a possible major metabolite of MTBE, was assessed in batch removal assays with BC-1. In this test, individual compounds were added (120-130 ppm) to one liter of BC-1 culture in a 1.5 liter vessel. Before addition of each compound, the culture was flushed with sterile 100% O₂ in a 1.5 liter sealed vessel for 2-5 minutes to achieve a dissolved oxygen level of 20 ppm. The reaction vessel was stirred continuously at 22-25BC and the depletion of substrates monitored by sampling (2-3 ml) over a 24 hours period. MTBE and TBA were analyzed by methods described below.

### Analysis of MTBE and TBA

Culture samples were analyzed for MTBE and t-butanol using a Hewlett-Packard Model 280 gas chromatography-flame ionization detection system. Compounds were separated on a Quadrex methyl silicone (1-Fm-thick film) capillary column having dimensions 25 m long and 0.025 mm inside diameter. Alltech/Applied Science Labs, State College, Pa.). The column was set initially at 30EC for 3 minutes and then programmed to 70EC at 20EC/min. The carrier gas consisted of helium (30 ml/min) and a N₂ make-up gas. One microliter split samples were analyzed. Retention times of TBA and MTBE were 3 and 3.8 min, respectively.

### Results of Substrate Removal Experiments

Results of batch substrate depletion assays with BC-1 in the presence of MTBE illustrated that MTBE (120 mg/liter) was rapidly degraded, within 4 hours at a rate of 34 mg/g of cells per hour. TBA was formed as a transient metabolic product of MTBE breakdown. The highest levels of TBA were reached after MTBE was completely utilized. TBA formed from MTBE declined at a slower rate (14 mg/g of cells per hour) than did MTBE. These results provide evidence that BC-1 degrades MTBE to TBA as a primary and transient intermediate.

### Part D; Oxygen Update Experiments

Oxygen uptake rates (OUR) were performed on the BC-1 culture in the presence of substrates and potential metabolic intermediates of MTBE. A Yellow Springs Instrument Company oxygen electrode-water bath assembly (Model 53; 5 ml reaction compartment) was used for these experiments. Suspended solids (TSS) from BC-1 were centrifuged (23,900×g, 10 min at 4° C.), resuspended to one-half the volume in a sterile phosphate-buffered saline solution, PBS (0.85% NaCl, 0.03M Na₂HPO₄ and 0.05M KH₂PO₄, pH 7.2). The 2× concentrated culture was aerated (sterile house air) continuously at 30° C. and maintained at a dissolved oxygen level of 6-7 ppm before using in OUR experiments. About 0.01-0.03g TSS were used in each reaction. Substrates were added at levels of 15 or 50 ppm from sterile stock (1,000 ppm) solutions and oxygen depletion monitored over 3-5 minutes at 30° C. The oxygen electrode and the dissolved oxygen concentration was interfaced and calibrated to the deflection of a 1 mV recorder (Houston Instrument Company) and rates calculated from the slopes of the tracings. OUR are given as mg oxygen utilized/g TSS/h.

The ability of BC-1 to oxidize MTBE and potential downstream degradation products and other cellular intermediates was determined by oxygen uptake rate (OUR) methods and these data are shown in Table 2. Highest OUR was observed with NH₄⁺, however, allylthiourea, a specific inhibitor of NH₄+oxidation, completely blocked this oxygen utilization. MTBE showed two distinct OUR, an initial faster (5.2-5.9 mg O₂/g/hr) and a slower (50% less) rate. Addition of allylthiourea had no effect on oxygen utilization in the presence of MTBE. t-Butylformate (TBF, t-butyl-COOH), an intermediate in the reaction of atmospheric-derived chloride and hydroxy free radicals with MTBE also enhanced oxygen uptake in BC-1. t-Butanol, isopropanol and lactate showed comparable OUR to MTBE (4.3-7 mg/g/h).

TABLE 2
Oxygen Uptake Rates (OUR) with Culture BC-1^{a)}

| | Net OUR |
|---|---|
| Substrate^{b)} | mgO₂/g TSS/h |
| NH₄⁺ | 17.4 |
| NH₄ + allylthiourea | −^{c)} |
| Allylthiourea | −^{c)} |
| MTBE | 5.2-5.9, 2.3^{d)} |
| MTBE + allythiorea | 5.2 |
| t-Buylformate (Na) | 7.2 |
| t-Butanol | 6.0 |
| Isopropanol | 4.3 |
| Lactate (Na) | 7.0 |

^{a)}Continuous culture treating high NH₄⁺ (120 ppm) and MTBE (150-200 ppm) levels.
^{b)}All compounds added at 50 ppm.
^{c)}Less than or equal to the endogeneous OUR.
^{d)}First and second OUR.

### Part E: Radiolabeled MTBE Experiments

The ¹⁴CH₃O-MTBE was custom synthesized by Amersham Corp., (Arlington Heights, Ill.). It had a specific activity of 1.19 F Ci/mg and was 99.3% pure by radiochromatography. Cultures were centrifuged, washed and resuspended in the same volume of sterile PBS buffer (PBS, 0.85% NaCl, 0.03 M Na₂HPO₄, 0.05 M KH₂PO₄, pH 7.2), and placed in 125 ml serum bottles sealed with Teflon® lined septa. ¹⁴CH₃O-MTBE was added to a concentration of 0.08 F Ci/ml and MTBE at 2 ppm. Cultures were incubated at 300 on a rotary shaker (150-200 rpm) for seven days. The amount of ¹⁴CO₂ formed was determined by placing a 10-ml aliquot of the culture in a similar serum bottle, adjusting the pH to ≦2 with 6N HCl and then flushing the bottle for one hour with a steady stream of N₂ into three gas washing bottles containing 0.1M Ba (OH)₂. The Ba¹⁴CO₃ precipitate (formed after co-precipitation with Na₂CO₃ addition) was collected onto 0.45 Fm Millipore filters, washed with PBS, dried and the radioactivity was counted. After removal of ¹⁴CO₂, the culture was filtered onto a 0.22 Fm Millipore filter, washed with PBS, dried and counted to estimate ¹⁴C activity incorporated into biomass (cells). The remaining radioactivity in the filtrate represents undegraded ¹⁴CH₃O-MTBE and/or ¹⁴C-metabolites. The efficiency of trapping ¹⁴CO₂ by this method was confirmed in separate experiments in which NaH¹⁴CO₃ was added (0.06 microwaves, 70 ppm as CO₂) to PBS or azide-inhibited cultures, acidified (pH≦2) and flushed into Ba(OH)₂ traps as described. The recovery of H¹⁴CO₃⁻ as Ba¹⁴CO₃ was 95-100% of the applied radioactivity. The ¹⁴C-radioactivity was determined by placing 1-ml amounts of culture fluid (total ¹⁴C) filtrates or filters containing Ba¹⁴CO₃ precipitates into glass scintillation vials containing 15 ml Aquasol-2 Universal 2SC Cocktail (NEN Dupont Research Products, Boston, Mass.). Vials were counted in a Packard TRI-CARB (Model 2500 TR) liquid scintillation analyzer (Packard Instrument Co., Meriden, Conn.).

Results of the biodegradation of radiolabeled ether (2 ppm) by BC-1 are given in Table 3. Less than 1% and 5% of the applied isotope was recovered as ¹⁴CO₂ and ¹⁴C-cells, respectively, in the abiotic (no culture) control and cultures containing the respiration inhibitor, sodium azide (2%). About 80% of the 14CH₃O-MTBE was incorporated into CO₂ and cells with the remainder (ca. 15%) as undegraded ether and/or ¹⁴C-metabolites. Addition of 100 ppm NH⁴₊ to metabolizing cultures had no competitive effect on stimulating or inhibiting MTBE biotransformation.

TABLE 3
Distribution of ¹⁴CH₃O-MTBE in Ether-Degrading Cultures

| | % of Applied ¹⁴CH₃O-MTBE^{a} in | | | |
|---|---|---|---|---|
| | | | MTBE &/or | % |
| Condition | CO₂ | Cells | Metabolites | Recovery |
| 1. Control (no cells) | 0.2 | 4.1 | 13.7 | 18 |
| 2. BC-1^{b} + Azide (2%) | 0.9 | 5.1 | 17.1 | 23.1 |
| 3. BC-1 | 39.0 | 42.1 | 17.8 | 98.9 |
| 4. BC-1 + NH₄^{+ (100 ppm)} | 42.3 | 40.3 | 12.5 | 95.1 |

^{a)}Mean of duplicate cultures did not differ by more than 10%.
^{b)}Suspended solids as TSS and VSS were 2440 and 1820 ppm, respectively.

### Chemicals

Common laboratory chemicals e.g. salts, bases acids, alcohols and ketones used were purchased from Mallinckrodt or Sigma Chemical Companies. MTBE and TBA were obtained as ≧98% pure material from Chem. Service Inc. of West Chester, Pa.

### Part F: Isolation of Pure Culture

Dilution enrichments of the present mixed culture were made to enhance isolation of a specific microbe degrading MTBE. In this method, the mixed culture BC-1 (ATCC No. 202057) enriched from activated sludge was used to isolate pure culture. The mixed bacterial culture BC-1 was prepared using the method described above. The MTBE and TBA degrading activity of BC-1 at 9° C. at 330 mg/L TSS is demonstrated in the following Table:

TABLE 4
Degradation of MTBE Utilizing Mixed Culture BC-1

MTBE Conc (ppm)

| Time (hr) | 6.5 | 14 | 19.2 | 40 | 100 | 190 |
|---|---|---|---|---|---|---|
| 0 | 6.5 | 14 | 19.2 | 40 | 100 | 190 |
| 0.5 | 5.5 | 10.8 | 19.2 | 37 | 100 | 190 |
| 1 | 4 | 9.4 | 15.4 | 30 | 90 | 197 |
| 4 | 2.1 | 5 | 14.1 | | 90 | |
| 5 | 1.2 | 5.4 | 11.2 | 30 | 82 | 176 |
| 7 | 0.7 | 3.5 | 4.4 | 19 | 82 | 178 |
| 24 | 0 | 0.44 | 0.27 | 8.8 | 53.3 | 150 |
| 48 | | 0 | 0.08 | 0.11 | 26.4 | 140 |
| 72 | | | 0 | 0 | 23.9 | 145 |
| 96 | | | | | 10.8 | 140 |
| 168 | | | | | 0 | 140 |
| 192 | | | | | | 127 |

TABLE 5
Degradation of TBA Utilizing Mixed Culture BC-1

| | TBA Conc (ppm) | | | | |
|---|---|---|---|---|---|
| Time (hr) | 1.1 | 2.6 | 5.6 | 14 | 28 |
| 0 | 1.1 | 2.6 | 5.6 | 14 | 28 |
| 0.5 | 1.2 | 2.7 | | | |
| 2 | 0.9 | 2.3 | 4.4 | 16 | 28 |
| 4 | 0.66 | 1.5 | | | |
| 6 | 0.47 | 1.4 | 4 | 11 | 26 |
| 24 | 0.094 | 0.26 | 1.1 | 5.9 | 17 |
| 48 | <0.01 | <0.01 | 0.024 | 0.27 | 10 |
| 72 | | | <0.01 | <0.01 | 3 |
| 144 | | | | | <0.1 |

Ten ml of the BC-1 was added to 10 ml sterile Difco Bushnell-Haas (MgSO₄, 200 mg/L; CaCl₂, 20 mg/L; KH₂PO₄, 1000 mg/L; K₂HPO₄ 1000 mg/L; NH₄NO₃ 1000 mg/L; FeCl₃, 50 mg/L, pH 7.0) minerals medium (3.5 g/L; referred to as BH) in stoppered serum bottles containing 1-5 mg/L MTBE. At weekly intervals, half of the culture volume (10 ml) was aseptically removed and 10 ml fresh sterile BH medium added to the remaining 10 ml of culture. The dilution enrichment method was continued for at least 2-3 months at 25° C. until a dilute suspension of bacteria degrading MTBE consistently degraded MTBE before each transfer interval. This dilution enrichment culture was subsequently streaked onto sterile Petri plates containing BH minerals plus 1.5% Difco Agar as solidifying agent. Plates were incubated at 25° C. or 30° C. and observed for the appearance of colonies after 3-5 days. Approximately 20 colonies were picked with sterile needles and inoculated into 20 serum vials containing sterile BH medium and 1-10 mg/L MTBE. These cultures were incubated at 25-30° C. and the loss of MTBE from the headspace of serum vials was determined. One isolate (10BC) completely degraded MTBE without the appearance of intermediates such as t-butyl alcohol.

### Part G. Degradation of MTBE by Pure Culture

The pure culture isolate was grown in R₂A broth medium (yeast extract 0.5 g/L; peptone 0.5 g/L; casein acid hydrolyzate, 0.5 g/L; soluble starch, 0.5 g/L; glucose 0.5 g/L; KH₂PO₄, 0.3 g/L; MgSO₄, 0.024 g/L; sodium pyruvate, 0.3 g/L; pH 7.0) for 24-48 hours at 25° C. The culture was then centrifuged (8000 rpm, 15 min.) and resuspended into 10 ml sterile phosphate-buffered saline (NaCl, 9 g/L; KH₂PO₄, 6.85 g/L; pH 7.0-7.2). The culture was transferred to a 30 ml serum vial. MTBE was added to a concentration of 5 mg/L and stoppered and sealed. The degradation of MTBE was followed at 25° C. over several days. Table 6 is an example of MTBE degraded by this pure culture from 5 mg/L to non-detectable concentrations (=5 μg/L) in 48 hours (Run #1). This culture was respiked with 5 mg/L MTBE. MTBE was degraded 95% (0.24 mg/L) in 27 hours (Run #2 Table 7).

TABLE 6
Run #1: Degradation of MTBE by Pure Culture

| Initial MTBE | MTBE after |
|---|---|
| mg/L | 48 hours |
| 5 | = 0.005 |

TABLE 7
Run #2: Degradation of MTBE by Pure Culture

| Initial MTBE | MTBE after |
|---|---|
| mg/L | 27 hours |
| 5 | 0.24 |

### Part H: Physiological Properties of Pure Culture

Table 8 summarizes some physiological properties and substrates utilized by one of the MTBE-degrading isolates, 10BC. The organism is an aerobic, morphologically irregular, gram-positive rod. The organism appears coccal-shaped when cultured on solid media. It grows on Tweens, dextrin, cellobiose, fructose, lactose, maltose, trehalose, glucose, adonitol, arabinose, lactose, sorbitol and acetate. 10BC also grows well on the metabolic intermediates in the MTBE pathway, namely, isopropanol, acetone and acetate. The culture grows well on a variety of complex bacteriological media including Trypticase Soy Broth and Agar (BBL, Becton Dickinson, Inc.) and Plate Count Agar (Difco). Based on physiological and biochemical features of 10BC as a non-fermentative gram-positive, oxidase-negative, catalase-negative bacterium and substrate utilization patterns in the Oxi/Ferm and Biolog assays characteristics described in Bergey's Manual of Systematic Bacteriology, it is probable that this isolate belongs to the family of organisms known as actinomycetes.

TABLE 8
Physiological features & substrates utilized
by pure culture isolate 10BC

| FEATURE | REACTION |
|---|---|
| Morpthology | Large rod (about 1.0 micron diam.), |
| | non-motile |
| Gram stain | Positive |
| Pigment formed | Orange (intracellular) |
| Oxidase reaction | Negative^{a)} |
| Optimum grow temp. | 20-35° C.; strict aerobe |
| Catalase | Negative |
| Feature | Tween 40, Tween 80, beta- |
| Substrates utilized in | cyclodextrin, dextrin, cellobiose, |
| Biolog assay^{b)} | fructose, lactulose, and trehalose |
| Substrates utilized | Glucose, adonitol, arabinose, |
| in Oxi/Ferm | lactose, sorbitol |
| Tube and Enterotube | |
| tests^{c)} | |
| Other substrates | Acetate, isopropanol, acetone, |
| utilized^{d)} | pentane, hexane, octane, decane, |
| | dodecane, tetradecane, hexadecane, |
| | maltose, mannitol, adipate, |
| | benzoate, citrate, lactate, tyrosine |
| Weak growth | MTBE, t-butyl alcohol, benzene, |
| substrates^{d)} | toluene, ethylbenzene, m-xylene |

^{a)Oxidase reaction negative when grown on most substrates.}
^{b)}Biolog Identification System, .GN and GP plates (Biolog, Inc; Hayward, CA) a carbon source utilization method for identification of gram-negative and gram-positive bacteria, Miller and Rhoden (J. Clin. Microbiol. 29(6):143-l47, 1991).
^{c)}Multimedia substrate utilization tubes for identification of oxidative and/or fermentative gam-negative rods (Hoffman-LaRoche, Inc.). Substrates tested in the Oxi/Ferm tube and Entertube include glucose, xylose, urea, citrate, arginine, lysine, lactose, sucrose, maltose, mannitol, dulcitol, phenylalanine and omitine.
^{d)}Substrates were added to sterile minerals Bushnell Haas medium or the Rowbotham and Cross basal medium (J. Gen. Microbiol. 100:231-240, 1977) at concentrations of 100-200 mg C/L (or 0.1-1% w/v for sugars, acids and hydrocarbons) and growth evaluated (visual) after inoculating media with 1 ml of acetate-grown (24 hour) and incubating cultures for 7-14 days at 30C.

### Part I: 16S rRNA Gene Sequence Analysis

The 16S rRNA gene of the 10BC pure culture was PCR amplified from genomic DNA isolated from 10BC bacterial colonies. Primers used for the amplification correspond to E. coli positions 005 and 1540 (full length packages) and 005 and 531 (500 bp packages). Amplification products were purified from excess primers and dNTPs using Microcon 100 (Amicon) molecular weight cut-off membranes and checked for quality and quantity by running a portion of the products on an agarose gel.

Cycle sequencing of the 16S rRNA amplification products was carried out using AmpliTaq FS DNA polymerase and dRhodamine dye terminators. Excess dye-labeled terminators were removed from the sequencing reactions using Sephadex G-50 spin column. The products were collected by centrifugation, dried under vacuum and frozen at −20° C. until ready to load. Samples were resuspended in a solution of formamide/blue dextran/EDTA and denatured prior to loading. The samples were electrophoresed on a ABI Prism 377 DNA Sequencer. Data was analyzed using PE/Applied Biosystem's MicroSeq™ microbial analysis and DNA editing and assembly software and database.

The top ten alignment matches below are presented in a percent genetic distance format. In this format a low percent indicates a close match.

Alignment: 504 base pairs 10BC

4.56% 504 Rhodococcus coprophilus

4.57% 503 Rodococcus rhodochrous

5.80% 500 Mycobacterium tokaiense

6.40% 500 Nocardia corynebacteroides

6.41% 499 Mycobacterium brumae

6.97% 502 Mycobacterium gadium

7.20% 500 Tsukamurella wratislaviensis

7.37% 502 Tsukamurella inchonensis

7.37% 502 Tsukamurella pulmonis

7.37% 502 Tsukamurella paurometabolum

The Neighbor joining (Saitou and Nei, Mol. Biol. Evol. 4(4):406-425, 1987) phylogenetic trees below are generated using the above top ten alignment matches.

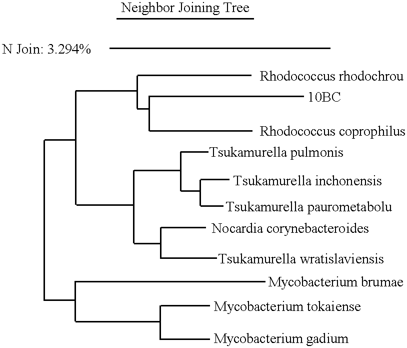

Concise alignments are also included below. These illustrate positions that differ between 10BC and the first match in the database. The position of the mismatch is read vertically from top to bottom and the sequences are read horizontally from left to right.

!Concise Alignment - 500 bp

| | |
|---|---|
| | \\111111222223444444444 |
| | 68777999012567333456668 |
| | 95389135913613578960130 |
| 10BC | ATGTCTCGTCGCCCTCCGGGGAC |
| Rhodococcus coprophilus | TCTATCTTATATTACGGATCCGT |

Data from the partial sequencing of 16S rRNA have enabled the supra generic relationship of actinomycetes to be established and this places Rhodococcus beside Mocardia and Mycobacterium among the nocardioform actinomycetes (Goodfellow, M 1989 Suprageneric classification of Actinomycetes in: Bergey's Manual of Systematic Bacteriology. Pp. 2333-2339. Holt, J. G. Ed. Williams & Williams. Baltimore, Md.)

### PART J: GC-FAME ANALYSIS

The strain was streaked onto trypticase soy agar [TSA]. The TSA plates were prepared for use in the GC-FAME analyses after 24 hour incubation. The strain was examined against both the Aerobe (TSBA [rev. 3.90]) and the Clinical Aerobe (CLIN [rev. 3.90]) databases. The strain was subsequently prepared for Biolog analysis by suspending it in sterile saline and loading the solution into the appropriate microtiter plates (Gram positive). The plates were incubated for 24 hours and then examined against version 3.5 of the Biolog™ database using an automated microplate reader.

TABLE 9
Summary of Results by GC-FAME and Biolog ™

| | Primary ID | | | Primary ID | | | |
|---|---|---|---|---|---|---|---|
| Strain | by | Sim. | Dist. | by | Plate | Sim. | Dist. |
| No. | GC-FAME | Coef | Coef | Biolog | Type | Coef | coef |
| 10BC | Rhodococcus | 0.291 | 5.925 | No ID | GP | 0.122 | 13.090 |
| | Rhodochrous | | | closest sp.: | | | |
| | [Clin] | | | Bacillus | | | |
| | | | | Brevis | | | |

### Part K: Degradation of MTBE with Pure Culture

Aqueous solutions containing various concentrations of MTBE: 5.7, 11.7, 20.9, 44.5, 90.2, 165, 350 ppm of MTBE were mixed with the present pure culture 10BC (2.76 g/L TSS at 25° C.) and incubated at 25° C. for various length of time and the concentrations of MTBE were measured. The results of the experiment is listed in Table 10 below.

TABLE 10

| | MTBE Conc (ppm) | | | | | | |
|---|---|---|---|---|---|---|---|
| Time (hr) | 5.7 | 11.7 | 20.9 | 44.5 | 90.2 | 165 | 350 |
| 0 | 5.7 | 11.7 | 20.9 | 44.5 | 90.2 | 165 | 350 |
| 1 | 5.6 | 11.6 | 20.1 | 45 | 82.5 | 153 | 308 |
| 2 | 5.7 | 10.7 | 18.5 | 49 | 86 | 160 | 310 |
| 5 | 5.3 | 10.3 | 17.9 | 48.6 | 82.5 | 174 | 296 |
| 7 | 6.7 | 8.3 | 13.2 | 40.6 | 78.8 | 136 | 330 |
| 12 | 0.34 | 0.39 | 0.9 | 8.2 | 31.1 | 111 | 241 |
| 14 | 0 | 0 | 0 | 1.37 | 9.75 | 42 | 21.9 |
| 24 | | | | 0 | 0 | 0 | 12.8 |

### Part L: Degradation of MTBE with Pure Culture

Aqueous solutions containing various concentrations of MTBE: 6.5, 14, 19.2, 40, and 100 of MTBE were mixed with the present pure culture 10BC (330 mg/L TSS at 9° C.) and incubated at 25° C. for various length of time and the concentrations of MTBE were measured. The results of the experiment is listed in Table 11 below.

TABLE 11

| | MTBE Conc (ppm) | | | | |
|---|---|---|---|---|---|
| Time (hr) | 1.1 | 2.6 | 5.6 | 14 | 28 |
| 0 | 6.5 | 14 | 19.2 | 40 | 100 |
| 0.5 | 5.5 | 10.8 | 19.2 | 37 | 100 |
| 1 | 4 | 9.4 | 15.4 | 30 | 90 |
| 4 | 2.1 | 5 | 14.1 | | 90 |
| 5 | 1.2 | 5.4 | 11.2 | 30 | 82 |
| 7 | 0.7 | 3.5 | 4.4 | 19 | 82 |
| 24 | 0 | 0.44 | 0.27 | 8.8 | 53.3 |
| 48 | | 0 | 0.08 | 0.11 | 26.4 |
| 72 | | | 0 | 0 | 23.9 |
| 96 | | | | | 10.8 |
| 168 | | | | | 0 |
| 192 | | | | | |

### Part M: Degradation of TBA with Pure Culture

Aqueous solutions containing various concentrations of t-butyl alcohol (TBA) were mixed with the present pure culture 10BC (820 mg/L TSS at 9° C.) and incubated at 25° C. for various length of time and the concentrations of TBA were measured. The results of the experiment is listed in Table 12 below.

| | Run #1 | Run #2 | Run #3 |
|---|---|---|---|
| Time (Day) | TBA mg/L | TBA mg/L | TBA mg/L |
| 0.00 | 3.3 | 6.3 | 21 |
| 0.04 | 3.6 | 6.3 | 19 |
| 0.21 | 3.4 | 6.4 | 20 |
| 1 | 3.3 | 5.3 | 16 |
| 2 | 1.1 | 1.5 | 0.98 |
| 3 | 0.26 | 0.29 | 0.028 |
| 4 | 0.076 | 0.063 | 0.005 |

### Part N: Effect of Storage Conditions (Temperature) on Rhodococcus sp (Strain 10BC) Activity for MTBE Degradation

A culture Medium (BHC₁₀)was prepared with BH+10 g/L Cerelose (1000 ml) at pH 7.2-7.4. BHC₁₀ was inoculated with 10-20 ml pure culture 10BC grown on BHC₁ (BH+1 g/L Cerelose)for 2-4 days at room temperature. Assay MTBE Design in die-away test system was conducted using the following procedure:

a) 10 ml culture is added to a 30 ml serum vial, sealed, and 5-8 ppm of MTBE (0.5 ml of 200 ppm sterile soln) was subsequently introduced. Analysis was conducted on MTBE in vials using calibrated Photovac in aliquots of 0.1 ml sample.

b) Assay culture at 0 hr. in duplicate prior to set up at 25° C., 4° C. and −70°°C.

c) Remove packed cell (pellet) aliquots at designated times; add 40 ml BH minerals solution; vortex well to suspend cells and remove 10 ml for die away test.

d) Determine Qmax mg MTBE/g TSS/hr.

The results of the study is summarized in Tables 13-16 below.

TABLE 13
MTBE DIE-AWAY (WASHED AND UNWASHED CULTURE)
MTBE ppm

| Culture | | | | | | | | |
|---|---|---|---|---|---|---|---|---|
| Condition | 0 h | 3 h | 24 h | 48 h | 74 h | 99 h | 120 h | 186 h |
| Unwashed | 8.76 | 8.57 | 8.68 | 9.10 | 8.40 | 8.71 | 8.23 | 8.75 |
| Washed (2×)* | 8.57 | 7.85 | .33 | 0 | | | | |
| (a) | | | | | | | | |
| Washed (2×)* | 8.09 | 7.98 | .39 | 0 | | | | |
| (b) | | | | | | | | |

*Washed culture was centrifuged and resuspended in BH minerals 2× before running die-way assay.

TABLE 14
MTBE DIE-AWAY (WASHED CELLS)
24 HR STORED SAMPLES:
MTBE PPM

| Storage | | | | |
|---|---|---|---|---|
| Culture | | | | |
| Condition ° C. | 0 hours | 3 hours | 24 h | 50 h |
| 25 | 8.28 | 8.32 | 0 | |
| 4 | 7.96 | 7.79 | .47 | 0 |
| −70 | 7.97 | 8.36 | .55 | 0 |

TABLE 15
MTBE DIE-AWAY (WASHED CELLS)
48 HR STORED SAMPLES:
MTBE PPM

| Storage | 7/16 | 7/16 | 7/17 |
|---|---|---|---|
| Culture | 3 pm | 6 pm | 5 pm |
| Condition ° C. | 0 h | 3 h | 20 h |
| 25 C. | 9.12 | 7.54 | 0 |
| 4 | 8.83 | 7.26 | 0 |
| −70 | 8.61 | 7.27 | 0 |

TABLE 16
MTBE DIE-AWAY (WASHED CELLS)
72 HR STORED SAMPLES:
MTBE PPM

| Storage | | | |
|---|---|---|---|
| Culture | | | |
| Condition ° C. | 0 hr | 24 hr | 46 hr |
| 25 C. | 8.51 | .42 | 0 |
| 4 | 8.62 | 0 | |
| −70 | 8.57 | 0 | |

Therefore, the above data show that the pure culture did not show any significant deterioration in MTBE activities after 72 hours of storage.

### Part O: Comparison of Degrading Activities in 10BC and ATCC 15998

The 10BC Culture of the present invention which belongs to Rhodococcus SP was compared with the pure culture with ATCC No. 15998 which is a Rhodococcus rhodochrous “ruber strain”

TABLE 17

| | MEDIUM/GROWTH | |
|---|---|---|
| CULTURE | CONDITIONS | INCUBATION/DAYS |
| 1. 10BC | UGA + .01% Tyrosine | 30° C., 5d |
| 2. 10BC | UGA + .1% Tyrosine | 30° C., 5d |
| 3. ATCC15998 | BHNPC10 (Cerelose | 30° C., 4d |
| | 10 g/L) | |
| 4. ATCC15998 | UGAC10 (Cerelose | 30° C., 6d |
| | 10 g/L) | |

One hundred milliliters of each of the cultures were centrifuged at 8000 rpm for 15 minutes and washed twice with lo sterile BH Mineral Solution. It was then resuspended to 10 ml BH and then sparge 20-30 seconds with 100% oxygen. Dispense 10 ml of the solution to 30 ml serium vials. The vials are sealed and about 10 ppm MTBE are added. MTBE die away test system was followed using the growth medium and conditions specified in Table 13 above. The results were analyzed with photovac and gas chromatography.

TABLE 18
MTBE CONDITIONS (DIE-AWAY) IN CULTURES
MTBE ppm

| Culture | 0 h | 3 h | 6 h | 25 h | 50 h |
|---|---|---|---|---|---|
| 10BC | 8.09 | 6.33 | 5.35 | 0.45 | 0 |
| (.01% Tyr) | | | | | |
| 10BC | 8.39 | 8.60 | 2.26 | 0 | — |
| (.1% Tyr) | | | | | |
| ATCC | 8.39 | 8.71 | 8.21 | 9.65 | 9.70 |
| 15998 | | | | | |
| BHNPC₁₀ | | | | | |
| ATCC | 8.32 | 8.65 | 8.78 | 9.45 | 9.83 |
| 15998 | | | | | |
| UGAC₁₀ | | | | | |

The results of the tests propose that the pure culture Rhodococcus rhodochrous “rubber strain” ATCC No. 15998 does not show the ability of degrading MTBE as demonstrated by the present pure culture.

### Part K: Induction of MTBE Biodegradation in 10BC

The 10BC culture was grown on a medium (100 ml) containing BH minerals (BH), 1 g/L ((NH₄)₂ SO₄, 1 g/L K₂HPO₄ and 10 g/L Cerelose (glucose) substrate. The culture was incubated on A shaker (200 rpm) at 30° C. for 48-72 hrs and then centrifuged at 8000 rpm for 15-20 minutes. The supernatant was decanted and the collected cells were washed twice in BH by the same centrifugation decanting procedure. After the final wash, the cell pellet is resuspended in 10 ml BH (w/o cerelose) and transferred first to a 30 ml serum bottle. The 20 ml headspace was fluidized with 100% O₂ and sealed with a butyl rubber stopper. MTBE was added in consecutive spikes at 10, 20, 40, 80 & 160 ppm and the biodegradation of MTBE by the culture was followed by taking a headspace sample (10-50 microliters) and determining the amount of MTBE using the Photovac GC (Model 10S Plus). In the induction method, doses of MTBE were added (from stock concentrated solutions of ether) in increasing concentrations after each previous dose was degraded e.g., after the 10 ppm dose degrades (e.g. 10-24 hr) then 20 ppm was added and the decline of MTBE in headspace was followed. This induction method is a method to induce the enzyme pathway in 10BC that is responsible for biodegradation of the ether.

TABLE 19
MTBE Biodegradation in 10BC Consecutive Spiking Experiment

| | Concentration |
|---|---|
| Time (Hours) | MTBE mg/L |
| 0 | 6.1 |
| 16 | 6.4 |
| 23 | 7.3 |
| 44 | 1.3 |
| 47 | 0.7 |
| 47 | 32.2 |
| 63 | 0 |
| 63 | 60.8 |
| 68 | 25.75 |
| 74 | 5.2 |
| 84 | 0 |
| 86.5 | 155 |
| 92.5 | 72.3 |
| 109.5 | 13.3 |
| 115.5 | 2 |
| 117.5 | 0.2 |
| 118 | 257 |
| 135.5 | 140 |
| 142 | 112 |
| 164 | 24.2 |
| 171.5 | 9.3 |
| 195.5 | 2.1 |

From the results of the above experiment, it can be seen that the MTBE degrading activity of the pure bacterial culture were induced to a higher level after the culture was exposed to MTBE for an extended period of time.

The ranges and limitations provided in the instant specification and claims are those which are believed to particularly point out and distinctly claim the instant invention. It is, however, understood that other ranges and limitations that perform substantially the same function in substantially the same manner to obtain the same or substantially the same result are intended to be within the scope of the instant invention as defined by the instant specification and claims.

What is claimed is:

1. A composition derived from an isolated mixed bacterial culture, wherein said culture is obtained by a process comprising the steps of:
adding an aqueous mixture comprising a first amount of activated sludge taken from a biotreater for treating wastewater in a chemical plant to a container,
adding a first portion of said branched alkyl ether to said container to obtain a first mixture, and
incubating said first mixture at a temperature from about 10° C. to about 60° C.,
wherein said culture can degrade at least 10% of MTBE in an aqueous mixture containing from about 0.01 ppm to about 5,000 ppm of MTBE to carbon dioxide within 70 hours.

2. A composition derived from an isolated mixed bacterial culture having an identifying characteristics of BC-1, ATCC No. 202057, wherein said identifying characteristics is the ability of aerobically degrading methyl-t-butyl ether (MTBE), added to the composition at a concentration of 0.01 to 500 ppm, to carbon dioxide within 70 hours.

3. The composition as described in claim 2, wherein said composition also degrades t-butyl alcohol.

4. The composition as described in claim 2, wherein said composition further comprises a mixed bacterial culture having an identifying characteristics of BC-1, ATCC No. 202057.

5. The composition as described in claim 2, wherein said composition degrades to carbon dioxide, MTBE and one or more of the following ether compounds: diisopropyl ether, ethyl-t-butyl ether, di-t-butyl ether, diisobutyl ether, isopropyl isobutyl ether, isopropyl t-butyl ether, t-amylmethyl ether, t-amylethyl ether, t-amyl ethyl ether, t-amyl propyl ether, t-amylisopropyl ether, t-amyl-n-butyl ether, t-amyl isobutyl ether, and t-amyl methyl ether within 70 hours.

6. The composition of claim 5, wherein the culture degrades at least 10% of the tertiary carbon-containing ether compounds in about 3 to 5 hours.

7. The composition of claim 2, wherein said composition derived from an isolated mixed bacterial culture having an identifying characteristics of BC-1, ATCC No. 202057 is a pure bacterial culture, which identifying characteristics being the ability of degrading methyl-t-butyl ether (MTBE) to carbon dioxide within 70 hours.

8. The composition as described in claim 7, wherein said composition has the identifying characteristics of ATCC No. ______.

9. The composition of claim 1, wherein said composition is a pure culture which degrades to carbon dioxide, MTBE and one or more of the following tertiary carbon-containing ether compounds: ethyl-t-butyl ether, t-amyl-n-butyl ether, t-amyl isobutyl ether, isopropyl t-butyl ether, t-amyl ethyl ether, t-amylpropyl ether, t-amylisopropyl ether, and methyl t-amyl ether within 70 hours.

10. A pure bacterial culture having the identifying characteristics of ATCC ______, wherein said identifying characteristics is the ability of degrading methyl-tert-butyl ether (MTBE) to carbon dioxide within 70 hours.

11. A pure bacterial culture belonging to the species of Rhodococcus. which degrades MTBE to carbon dioxide within 70 hours.

12. The pure bacterial culture as described in claim 11, which culture also degrades t-butyl alcohol.

13. A process for degrading MTBE in a MTBE-containing mixture, which process comprises growing in the presence of said MTBE-containing mixture a composition derived from an isolated mixed bacterial culture, wherein said mixed bacterial culture is obtained by a process comprising the steps of:
adding an aqueous mixture comprising a first amount of activated sludge taken from a biotreater for treating wastewater in a chemical plant to a container,
adding a first portion of said branched alkyl ether to said container to obtain a first mixture, and
incubating said first mixture at a temperature from about 10° C. to about 60° C.,
wherein said culture can degrade at least 10% of MTBE in an aqueous mixture containing from about 0.01 ppm to about 5,000 ppm of MTBE to carbon dioxide within 70 hours.

14. A process for treating groundwater, wastewater or soil containing an ether selected from the group consisting of MTBE, diisopropyl ether, ethyl-t-butyl ether, di-t-butyl ether, disobutyl ether, isopropyl isobutyl ether, isopropyl t-butyl ether, t-amyl methyl ether, t-amyl ethyl ether, t-amyl propyl ether, t-amylisopropyl ether, t-amyl-n-butyl ether, t-amyl isobutyl ether, and t-amyl methyl ether, to reduce the ether content thereof, which process comprises growing in the presence of said ground-water or wastewater under aerobic conditions (1) a pure bacterial culture having the identifying characteristics of ATCC No. ______ and (2) a mixed bacterial culture having the identifying characteristics of BC-1, ATCC No. 202057.

15. A process for degrading MTBE in a MTBE-containing mixture, which process comprises growing in the presence of said MTBE-containing mixture a composition derived from an isolated mixed bacterial culture, which degrades at least 10% of the methyl t-butyl ether (MTBE) in an aqueous mixture containing from 0.01 to 5000 ppm of MTBE to carbon dioxide in 70 hours, wherein said mixed culture is isolated from a mixed bacterial culture obtained by a process comprising the steps of:
adding an aqueous mixture comprising a first amount of activated sludge taken from a biotreater for treating wastewater in a chemical plant to a container,
adding a first portion of MTBE to said container to obtain a first mixture which contains from about 10 mg to about 500 mg of MTBE, incubating said first mixture at a temperature from about 10° C. to about 60° C., periodically adding additional amounts of the biosludge to said container,
periodically withdrawing from the container from about 10% to about 70% of the supernatant medium followed by adding mineral solution to replace the supernatant withdrawn, and periodically adding MTBE to the container in an amount sufficient to maintain the concentration of MTBE in culture in the container at from about 10 mg to about 500 mg.

16. A process for degrading a branched ether selected from a group consisting of MTBE, diisopropyl ether, ethyl-t-butyl ether, di-t-butyl ether, disobutyl ether, isopropyl isobutyl ether, isopropyl t-butyl ether, t-amyl methyl ether, t-amyl ethyl ether, t-amyl propyl ether, t-amylisopropyl ether, t-amyl-n-butyl ether, t-amyl isobutyl ether, and t-amyl methyl ether in an ether-containing mixture, which process comprises growing in the presence of said ether-containing mixture a pure bacterial culture isolated from a mixed bacterial culture obtained by a process comprising the steps of:
adding a solution comprising a first amount of activated sludge taken from a biotreater for treating wastewater in a chemical plant to a container,
adding a first portion of [a] said branched alkyl ether to said container to obtain a first mixture, and
incubating said first mixture at a temperature from about 10° C. to about 60° C.;
wherein said culture can degrade at least 10% of MTBE in an aqueous mixture containing from about 0.01 ppm to about 5,000 ppm of MTBE to carbon dioxide within 70 hours.

17. The process as described in claim 16, wherein said pure bacterial culture has an identifying characteristics of ATCC No. ______ which degrades MTBE to carbon dioxide in 70 hours.

18. A process for degrading t-butyl alcohol (TBA) in a TBA-containing mixture, which process comprises growing in the presence of said TBA-containing mixture a pure bacterial culture capable of degrading both TBA and methyl-t-butyl ether (MTBE).

19. The process as described in claim 18, wherein said culture is capable of degrading MTBE to carbon dioxide in 70 hours and said process further comprising growing in the presence of said TBA-containing mixture a mixed bacterial culture having the identifying characteristics of BC-1 ATCC NO. 202057, in addition to said pure bacterial culture.
